(19) United States
(12) Patent Application Publication
(10) Pub. No.: US 2019/0054270 A1
(43) Pub. Date: Feb. 21, 2019

(54) ANTI-OCCLUSION CATHETER

(71) Applicant: Becton, Dickinson and Company, Franklin Lakes, NJ (US)

(72) Inventor: Stephen Thomas Bornhoft, Raynham, MA (US)

(21) Appl. No.: 15/677,847

(22) Filed: Aug. 15, 2017

(51) Int. Cl. A61M 25/00; A61M 39/10

(52) U.S. Cl. CPC A61M 25/0052; A61M 25/065; A61M 39/10

(57) ABSTRACT

An intravenous catheter may include a distal end, a proximal end, a lumen extending between the distal end and the proximal end, and an inner surface forming the lumen. At least a portion of the inner surface may include a non-circular and non-oval cross-section, which may prevent occlusion of the intravenous catheter when a kink in the intravenous catheter occurs. The non-circular and non-oval cross-section may include a variety of shapes. For example, the non-circular and non-oval cross-section may be polygonal and/or include one or more ribs.

### BACKGROUND OF THE INVENTION

Catheters may be used for infusion. For example, catheters may be used to infuse normal saline solution, various medicaments, or total parenteral nutrition into a patient. Catheters may also be used for collecting blood from the patient.

Catheters are often inserted into vasculature of the patient for infusion and/or collection as part of an intravenous (IV) catheter assembly. The catheter assembly generally includes a catheter adapter that supports the catheter. The catheter adapter may be coupled to a needle hub, which may support an introducer needle. The needle may be positioned within the catheter such that a beveled portion of the needle is exposed beyond a tip of the catheter for insertion into the vasculature.

The needle and catheter are generally inserted through skin of the patient and into the vasculature at an angle of about 30°. Once the needle and catheter tip enter a blood vessel of the patient, the needle and catheter may then be repositioned so that the needle and catheter are brought into a position generally parallel with the blood vessel. Following insertion and placement of the catheter in the blood vessel, the needle may be removed from the catheter, and the catheter may provide IV access to the patient. The catheter adapter may also be secured to the patient to prevent premature removal of the catheter.

Typically the catheter adapter is secured to the patient by fastening the catheter adapter to the skin via tape or another adhesive. In securing the catheter adapter to the skin, a portion of the catheter may arch to accommodate a transition from the generally parallel, secured catheter adapter, to an insertion angle of the catheter, which may be about 30°. A clinician may ensure that an extended section of catheter is left between the patient and the catheter adapter to allow for transitional arching of the catheter.

The portion of the catheter disposed proximate and distal to the distal end of the catheter may be referred to in the present disclosure as the “root region” of the catheter. In some instances, the root region may correspond to a region of the catheter between the skin of the patient and the distal end of the catheter adapter when the catheter is inserted into the blood vessel of the patient. The root region may be susceptible to kinking or occlusion when the catheter assembly is inserted into the blood vessel of the patient due to the transitional arching of the catheter.

When the catheter adapter is secured to the skin, the root region of the catheter may experience leverage forces. In further detail, the catheter may act as a lever and a distal end of the catheter adapter may act as a fulcrum exerting an upward force on the root region of the catheter. This upward force of the distal end of the catheter adapter, which may be more rigid than the catheter, may result in an increased likelihood of kinking and occlusion of the root region. The kinking of the catheter may serve to slow or stop flow through the catheter thereby inhibiting the function of the catheter assembly and creating undesirable backpressures. Occlusion resulting from the kinking may reduce the efficiency of the catheter assembly and prevent infusates from being delivered to the patient. Therefore, a need exists for devices, systems, and methods that reduce a risk of catheter occlusion.

### BRIEF SUMMARY OF THE INVENTION

The present disclosure relates generally to a catheter and associated systems and methods. In some embodiments, the catheter may be an IV catheter used for infusion and/or blood collection. In some embodiments, the catheter may include a distal end, a proximal end, a lumen extending between the distal end and the proximal end, and an inner surface forming the lumen. In some embodiments, at least a portion of the inner surface may include a non-circular and non-oval cross-section, which may prevent occlusion of the catheter when a kink in the catheter occurs. In further detail, in some embodiments, the non-circular and non-oval cross-section may facilitate an incomplete seal at the kink in the catheter, allowing fluid to flow through the catheter when the catheter is kinked. In some embodiments, the incomplete seal may allow the kink in the catheter to be undone as fluid flowing through the kink creates enough pressure to unkink the catheter.

In some embodiments, the inner surface of the catheter may include the non-circular and non-oval cross-section along all or a portion of a length of the catheter. Thus, in some embodiments, at least a portion of the inner surface may be non-cylindrical. The non-circular and non-oval cross-section may include any number of shapes and configurations that allow fluid to flow through the catheter when the catheter is kinked. As an example, the non-circular and non-oval cross-section may be polygonal. In further detail, in some embodiments, the non-circular and non-oval cross-section may be triangular, square, pentagonal, hexagonal, heptagonal, octagonal, etc. As another example, the non-circular and non-oval cross-section may include one or more ribs and/or one or more grooves.

In some embodiments, the one or more ribs and/or the one or more grooves may be oriented in a direction generally parallel with a longitudinal axis of the catheter. In these embodiments, the one or more ribs and/or the one or more grooves may extend along the inner surface of the catheter generally parallel to the longitudinal axis of the catheter.

In some embodiments, the catheter may be included in a catheter assembly, which may include various other components. In some embodiments, the catheter assembly may include a catheter adapter. In some embodiments, the catheter may extend distally from a distal end of the catheter adapter.

In some embodiments, the root region or a portion of the inner surface corresponding to a portion of the catheter disposed proximate and distal to the distal end of the catheter adapter may include the non-circular and non-oval cross-section, which may decrease the susceptibility of the root region to kinking and occlusion. In some instances, the root region may correspond to a region of the catheter between the skin of the patient and the distal end of the catheter adapter when the catheter is inserted into the blood vessel of the patient.

### BRIEF DESCRIPTION OF THE DRAWINGS

In order that the manner in which the above-recited and other features and advantages of the invention will be readily understood, a more particular description of the catheter briefly described above will be rendered by reference to specific embodiments thereof, which are illustrated in the appended Figures. Understanding that these Figures depict only typical embodiments and are not, therefore, to be considered to be limiting of its scope, the invention will be described and explained with additional specificity and detail through the use of the accompanying Figures in which:

FIG. 1A is an upper perspective view of an example catheter assembly, illustrating the catheter assembly in an insertion configuration, according to some embodiments;

FIG. 1B is a side view of the catheter assembly of FIG. 1A, illustrating the catheter assembly in the insertion configuration, according to some embodiments;

FIG. 1C is an enlarged upper perspective view of a portion of the catheter assembly of FIG. 1A, illustrating an example introducer needle extending through an example catheter, according to some embodiments;

FIG. 2 is a cross-sectional view of the catheter of the catheter assembly of FIG. 1A, wherein the catheter includes a non-circular and non-oval inner surface, according to some embodiments;

FIG. 3 is a cross-sectional view of the catheter of the catheter assembly of FIG. 1A along line 3-3 of FIG. 1A, wherein the catheter includes the non-circular and non-oval inner surface, according to some embodiments;

FIG. 4 is a cross-sectional view of the catheter of the catheter assembly of FIG. 1A along line 3-3 of FIG. 1A, wherein the catheter includes another non-circular and non-oval inner surface, according to some embodiments;

FIG. 5 is a cross-sectional view of the catheter of the catheter assembly of FIG. 1A along line 3-3 of FIG. 1A, wherein the catheter includes another non-circular and non-oval inner surface, according to some embodiments;

FIG. 6 is an upper perspective view of an example catheter tip of the catheter assembly of FIG. 1A, according to some embodiments; and

FIG. 7 is an upper perspective view of another example catheter tip of catheter assembly of FIG. 1A, according to some embodiments.

### DETAILED DESCRIPTION OF THE INVENTION

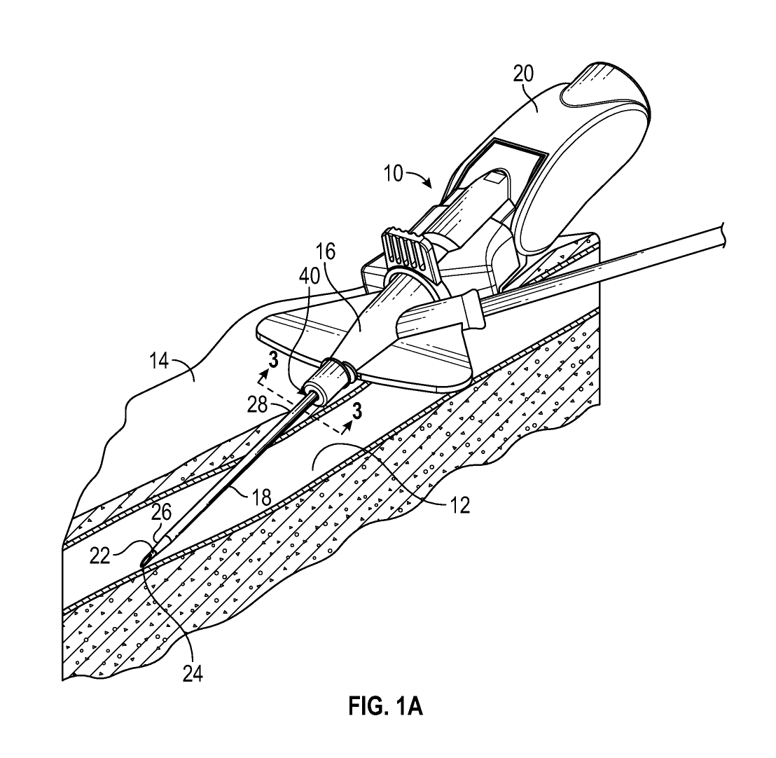

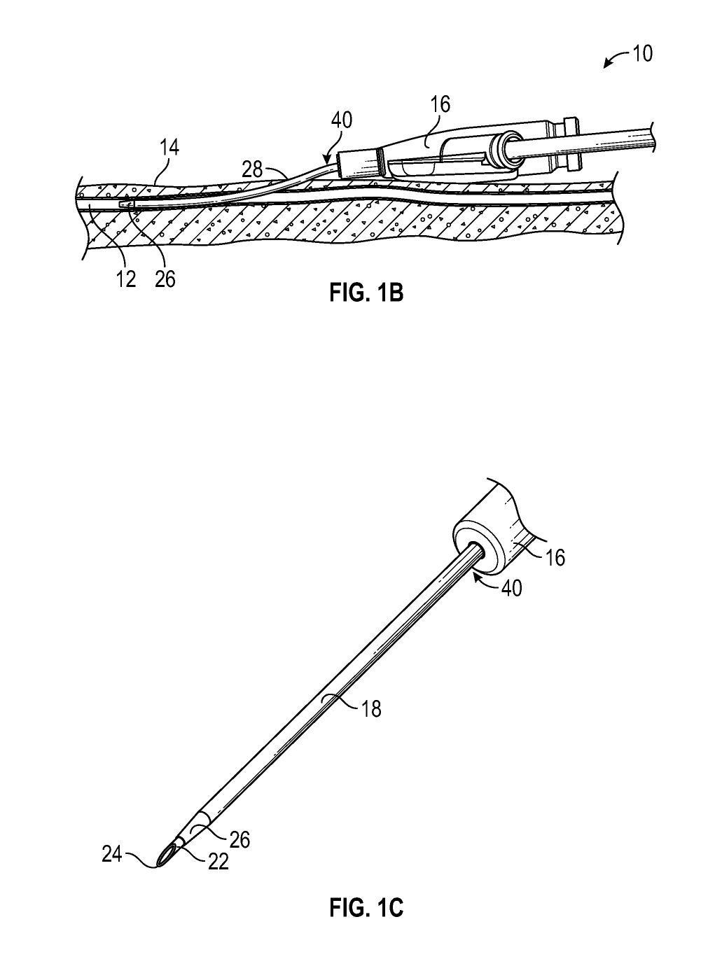

The presently preferred embodiments of the described invention will be best understood by reference to FIGS. 1-7, wherein like parts are designated by like numerals throughout. It will be readily understood that the components of the present invention, as generally described and illustrated in FIGS. 1-7 in the present disclosure, could be arranged and designed in a wide variety of different configurations. Thus, the following more detailed description, as represented in FIGS. 1-7, is not intended to limit the scope of the invention, as claimed, but is merely representative of some embodiments of the invention.

Referring now to FIGS. 1A-1C, a catheter assembly 10 is illustrated, according to some embodiments. The catheter assembly 10 may provide access to vasculature 12 of a patient via skin 14 of the patient. In some embodiments, the catheter assembly 10 may include a catheter adapter 16, which may support a catheter 18. In some embodiments, the catheter 18 may include a tube. In some embodiments, the catheter 18 may extend outwardly from catheter adapter 16. In some embodiments, a fluid pathway of the catheter assembly 10 may extend through the catheter adapter 16 and the catheter 18. Thus, in some embodiments, the catheter 18 may be in fluid communication with the catheter adapter 16.

It is understood that the catheter 18 may be used in various types of catheter assemblies and/or vascular access devices. In some embodiments, the catheter assembly 10 may correspond to the BD INTIMA II™ IV Catheter, the BD NEXIVA™ DIFFUSICS™ Closed IV Catheter System, the BD INSYTE™ AUTOGUARD™ BC Shielded IV Catheter, or another catheter assembly.

In some embodiments, the catheter assembly may include any number of components. In some embodiments, the catheter assembly 10 may include a needle hub 20, which may support an introducer needle 22. In some embodiments, the introducer needle 22 may extend through the catheter adapter 16 and the catheter 18 such that a beveled tip 24 of the introducer needle 22 extends beyond a distal end 26 or catheter tip of the catheter 18. In some embodiments, the beveled tip 24 may provide a cutting surface whereby to penetrate the skin 14 and provide access to the vasculature 12. In some embodiments, once the catheter 18 is inserted into the vasculature 12 via an insertion site 28 in the skin 14, the introducer needle 22 may be withdrawn from the vasculature 12 and/or removed from the catheter 18, thereby providing intravenous access to the patient via the catheter 18 and the catheter adapter 16.

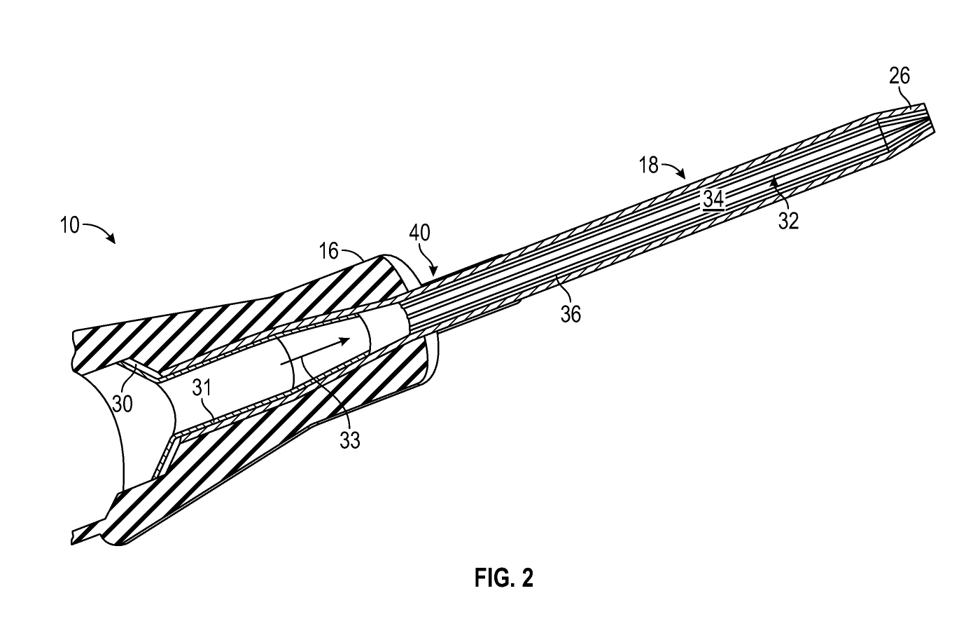

Referring now to FIG. 2, in some embodiments, the catheter 18 may include the distal end 26, a proximal end 30, a lumen 32 extending between the distal end 26 and the proximal end 30, and an inner surface 34 forming the lumen 32. In some embodiments, at least a portion of the inner surface 34 may include a non-circular and non-oval cross-section, which may prevent occlusion of the catheter 18 when a kink in the catheter occurs. In further detail, in some embodiments, the non-circular and non-oval cross-section may facilitate an incomplete seal at the kink in the catheter 18, allowing fluid to flow through the fluid pathway 33 when the catheter 18 is kinked. In some embodiments, the incomplete seal may allow the kink in the catheter 18 to be undone as fluid flowing through the kink creates enough pressure to unkink the catheter 18.

In some embodiments, the inner surface 34 of the catheter 18 may include the non-circular and non-oval cross-section along all or a portion of a length of the catheter 18. Thus, in some embodiments, at least a portion of the inner surface 34 may be non-cylindrical. In some embodiments, the portion of the inner surface 34 that is non-cylindrical may include or correspond to the root region 40.

In some embodiments, the non-circular and non-oval cross-section may extend at least through the root region 40, as illustrated, for example, in FIG. 2. The root region 40 may be susceptible to kinking or occlusion when the catheter assembly 10 is attached to the skin 14 and inserted into the vasculature 12 of the patient due to arching of the catheter to accommodate a transition from the generally parallel, secured catheter adapter, to an insertion angle of the catheter, which may be about 30°. In some embodiments, the circular and non-oval cross-section may extend along another portion of the catheter 18 and/or an entire length of the catheter 18.

The catheter 18 may be coupled to the catheter adapter 16 in various ways. In some embodiments, the catheter 18 may be mechanically attached to the catheter adapter 16 via insertion of a tubing insert 31 into a distal end of the catheter adapter 16. In some embodiments, the tubing insert 31 may secure the catheter 18 within a lumen of the catheter adapter 16 in a fluidtight manner.

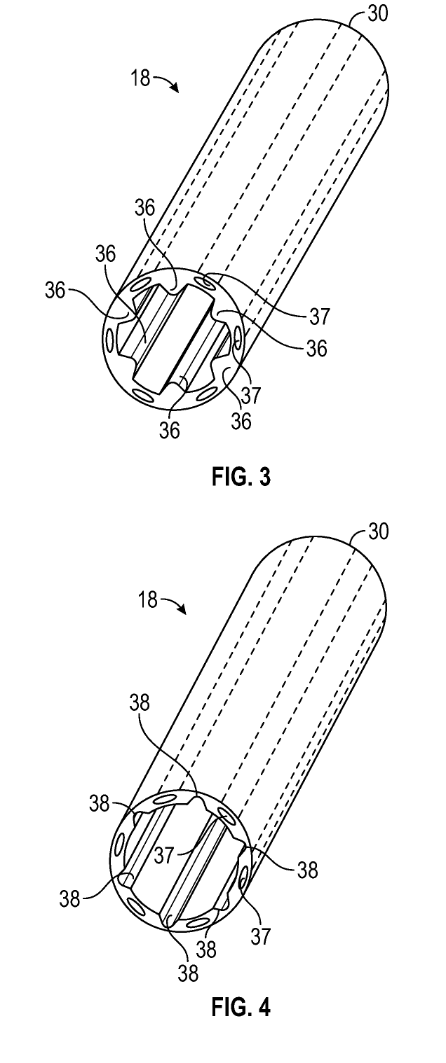

The non-circular and non-oval cross-section may include any number of shapes and configurations that allow fluid to flow through the catheter 18 when the catheter 18 is kinked. In some embodiments, the non-circular and non-oval cross-section may be symmetric. In other embodiments, the non-circular and non-oval cross-section may be asymmetric. Referring now to FIGS. 2-3, in some embodiments, the non-circular and non-oval cross-section may include one or more ribs 36, as illustrated, for example, in FIG. 2. In some embodiments, the ribs 36 may be oriented in a direction generally parallel with a longitudinal axis of the catheter 18. In some embodiments, the ribs 36 may extend along the inner surface 34 of the catheter 18 generally parallel to the longitudinal axis of the catheter 18.

In some embodiments, the ribs 36 may be spaced apart about a circumference of the inner surface 34. In some embodiments, the ribs 36 may be evenly spaced apart about the circumference of the inner surface 34. In some embodiments, the inner surface 34 may include any number of ribs 36. In some embodiments, the inner surface 34 may include two, three, four, five, six, seven, eight, nine, ten, or more ribs 36. In some embodiments, the ribs 36 may extend along the inner surface 34 of the root region 40. In some embodiments, the ribs 36 may extend along another portion of the inner surface 34 of the catheter 18 and/or along the entire length of the catheter 18.

Referring now to FIG. 3, in some embodiments, the catheter 18 may include radiopaque material contained within a wall of the catheter 18. In some embodiments, the radiopaque material may include radiopaque strips 37, which may run lengthwise within the wall of the catheter 18. In some embodiments, the radiopaque strips 37 may be incorporated into the catheter 18 to enhance the visibility of the catheter 18 in an x-ray.

Referring now to FIG. 4, in some embodiments, the non-circular and non-oval cross-section may include one or more grooves 38, as illustrated, for example, in FIG. 4. In some embodiments, the grooves 38 may be oriented in a direction generally parallel with a longitudinal axis of the catheter 18. In some embodiments, the grooves 38 may extend along the inner surface 34 of the catheter 18 generally parallel to the longitudinal axis of the catheter 18.

In some embodiments, the grooves 38 may be spaced apart about a circumference of the inner surface 34. In some embodiments, the grooves 38 may be evenly spaced apart about a circumference of the inner surface 34. In some embodiments, the inner surface 34 may include any number of grooves 38. In some embodiments, the inner surface 34 may include two, three, four, five, six, seven, eight, nine, ten, or more grooves 38. In some embodiments, the grooves 38 may extend along the inner surface 34 of the root region 40. In some embodiments, the grovoes 38 may extend along another portion of the inner surface 34 of the catheter 18 and/or along the entire length of the catheter 18.

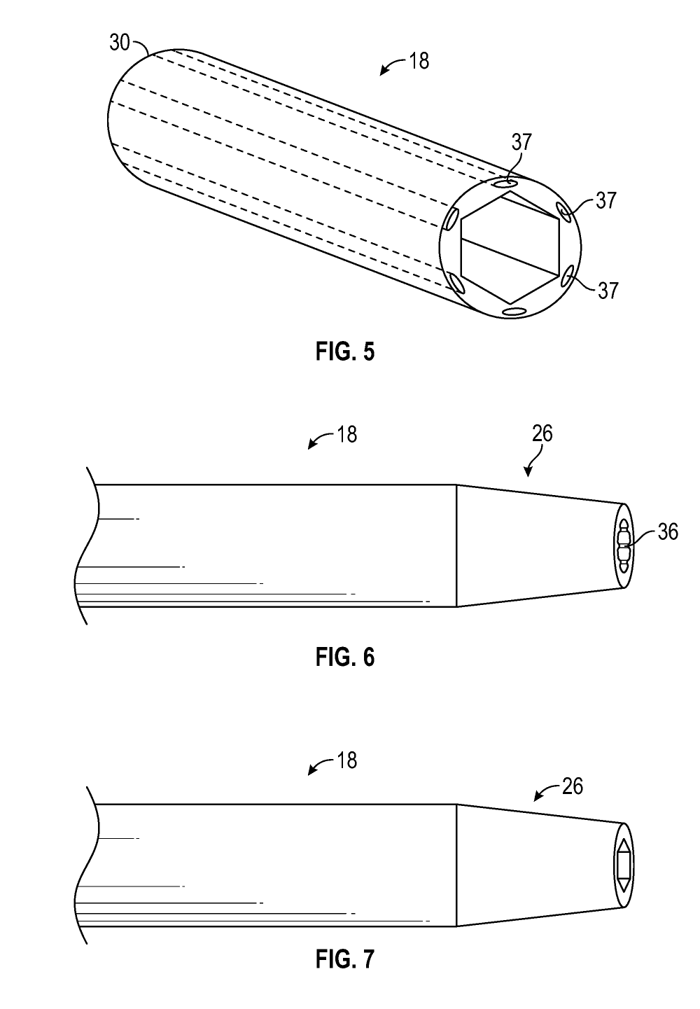

Referring now to FIG. 5, in some embodiments, the non-circular and non-oval cross-section may be polygonal. For example, the non-circular and non-oval cross-section may be triangular, square, pentagonal, hexagonal, heptagonal, octagonal, etc. Thus, in some embodiments, at least a portion of the inner surface may be polygon-shaped. In some embodiments, the polygon shape may extend through the root region 40. In some embodiments, the polygon shape may extend through another portion of the catheter 18 and/or the entire length of the catheter 18.

Referring now to FIGS. 6-7, in some embodiments, the non-circular and non-oval cross-section may extend through the distal end 26 of the catheter 18. In some embodiments, the non-circular and non-oval cross-section may not extend through the distal end 26 of the catheter 18. In some embodiments, the distal end 26 may include various shapes. In some embodiments, the inner surface 34 and/or an outer surface of the distal end 26 may be tapered, as illustrated, such as, for example, in FIGS. 6-7.

The present invention may be embodied in other specific forms without departing from its structures, methods, or other essential characteristics as broadly described herein and claimed hereinafter. It is also understood that any number of protrusions, ribs, grooves, shapes, and configurations may be combined to form the non-circular and non-oval cross-section and the non-cylindrical inner surface. The described embodiments and examples are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

1. An intravenous catheter comprising a distal end, a proximal end, a lumen extending between the distal end and the proximal end, and an inner surface forming the lumen, wherein at least a portion of the inner surface comprises a non-circular and non-oval cross-section.

2. The intravenous catheter of claim 1, wherein the non-circular and non-oval cross-section is polygonal.

3. The intravenous catheter of claim 1, wherein the non-circular and non-oval cross-section comprises one or more ribs.

4. The intravenous catheter of claim 3, wherein the one or more ribs extend along the inner surface of the catheter generally parallel to a longitudinal axis of the intravenous catheter.

5. The intravenous catheter of claim 1, wherein the non-circular and non-oval cross-section comprises one or more grooves.

6. The intravenous catheter of claim 5, wherein the one or more grooves extend along the inner surface of the catheter generally parallel to a longitudinal axis of the intravenous catheter.

7. The intravenous catheter of claim 1, wherein the intravenous catheter is configured to be coupled to and extend distally from a catheter adapter, wherein a portion of the inner surface corresponding to a portion of the intravenous catheter configured to be disposed proximate and distal to a distal end of the catheter adapter comprises the non-circular and non-oval cross-section.

8. The intravenous catheter of claim 1, wherein the non-circular and non-oval cross-section is asymmetric.

9. The intravenous catheter of claim 1, wherein the non-circular and non-oval cross-section is symmetric.

10. An intravenous catheter assembly, comprising:
a catheter adapter;
a catheter extending distally from the catheter adapter, wherein the catheter comprises a distal end, a proximal end, a lumen extending between the distal end and the proximal end, and an inner surface forming the lumen, wherein at least a portion of the inner surface comprises a non-circular and non-oval cross-section.

11. The intravenous catheter assembly of claim 10, wherein a portion of the inner surface corresponding to a portion of the intravenous catheter disposed proximate and distal to a distal end of the catheter adapter comprises the non-circular and non-oval cross-section.

12. The intravenous catheter assembly of claim 10, wherein the non-circular and non-oval cross-section is polygonal.

13. The intravenous catheter assembly of claim 10, wherein the non-circular and non-oval cross-section comprises one or more ribs.

14. The intravenous catheter assembly of claim 13, wherein the one or more ribs are oriented in a direction generally parallel with a longitudinal axis of the catheter.

15. The intravenous catheter assembly of claim 10, wherein the non-circular and non-oval cross-section comprises one or more grooves.

16. The intravenous catheter assembly of claim 15, wherein the one or more grooves are oriented in a direction generally parallel with a longitudinal axis of the catheter.

17. An intravenous catheter assembly, comprising:
a catheter adapter;
a catheter extending distally from the catheter adapter, wherein the catheter comprises a distal end, a proximal end, a lumen extending between the distal end and the proximal end, and an inner surface forming the lumen, wherein at least a portion of the inner surface corresponding to a portion of the catheter disposed proximate and distal to the catheter adapter is non-cylindrical.

18. The intravenous catheter assembly of claim 17, wherein at least the portion of the inner surface is polygon-shaped.

19. The intravenous catheter assembly of claim 17, wherein at least the portion of the inner surface comprises a plurality of ribs, wherein the ribs are spaced apart about a circumference of the inner surface.

20. The intravenous catheter assembly of claim 17, wherein at least the portion of the inner surface is hexagon shaped.
